(19) United States
(12) Patent Application Publication
(10) Pub. No.: US 2008/0097406 A1
(43) Pub. Date: Apr. 24, 2008

(54) System and method of using a color-coded medical apparatus

(76) Inventor: Eleanor A. Freed, Gilbertsville, PA (US)

Correspondence Address: WOLF, BLOCK, SCHORR & SOLIS-COHEN LLP, 1650 ARCH STREET, 22ND FLOOR, PHILADELPHIA, PA, 19103-2334, US

(21) Appl. No.: 11/505,756

(22) Filed: Aug. 17, 2006

(51) Int. Cl. A61M 25/00

(52) U.S. Cl. 604/533

(57) ABSTRACT

Color-coded medical apparatus, system and method for the administration of fluids. The tubing has a distal end and a body and contains a visible marking for identification of the fluid being administered from an intravenous bag through the tubing. The tubing and intravenous bag may be color-coded a predetermined color. The color is used to differentiate one fluid from another. Likewise, a colored collar can be used to engage pre-existing tubing in order to differentiate one fluid from another. The system for the administration of fluids incorporates at least one color-coded tube and intravenous bag of a first color and a second color-coded tube and intravenous bag of a second color, allowing one to differentiate between a first fluid and a second fluid by correlating the first color with the second color.

### FIELD OF INVENTION

The present invention relates generally to the field of medical supplies, and specifically to color-coded medical tubing and related accessories.

### BACKGROUND OF INVENTION

Tubing, as well as intravenous or IV bags in the medical industry have generally been made of clear polymer or rubber. This type of tubing along with intravenous bags are normally used to provide intravenous medication or medical fluids to patients when used in connection with a peripheral or venous catheter inserted into the patient's veins or arteries.

Different types of medical fluids are used for intravenous drips, which incorporate medical tubing and intreavenous bags. Typically, the fluids contained in an invtravenous bag are aqueous solutions of mineral salts or other water-soluble molecules, i.e., crystalliods. The most common of these is normal saline, which is an aqueous solution of sodium chloride (at a 0.9% concentration). Another fluid is Ringer's lactate or Ringer's solution, an isotonic solution often used for mass fluid replacement. Yet another fluid is D5W, an aqueous solution of 5% dextrose, which is used in place of Ringer's solution or saline when a patient is at risk of having high blood pressure or low blood sugar. Other fluids contain larger insoluble molecules such as gelatin or blood, i.e., colloids.

Often times, more than one intravenous tube is used on a single patient, where the multiple intravenous tubes often become twisted, intertwined and tangled with one another. This makes it difficult for medical personnel to determine the specific medication flowing through the tube. This effect is more pronounced during emergency situations when the speed of giving medication and medical aid to a patient is crucial to the patient's survival. The use of multiple intravenous tubes increases the risk to a patient due to the potential difficulty in quickly identifying the particular medication or type of medication being administered to the patient. Moreover, The risk also increases as, at often times, multiple does of medication are administered throughout the day.

In addition, medical personnel such as doctors, nurses and aids regularly interact with the same patient. Such medical personnel may have to add additional amounts of medication or infuse additional fluids, remove intravenous lines from the patient, or a combination thereof. Thus, the medical personnel have to, in short time, familiarize themselves with the current state of the patient as well as the medication currently being administered to the patient. This increases the risk of misidentifying a medication or a medical fluid contained in a tube and intravenous bag. This risk is heightened when such medical personnel unfamiliar with the current medication being administered to the patient (in addition to the patient's current state) have to quickly diagnose and/or react to a drastic change in the state of a patient, for example, when a patient suddenly goes into cardiac arrest.

Accordingly, there is a need in the art to differentiate between varying types of medication or medical fluid infused into a patient from an intravenous line. There is also a need in the art to implement a system that is capable of immediately differentiating between varying types of medication or medical fluid infused into a patient that can incorporate currently existing and ubiquitous devices.

### SUMMARY OF INVENTION

These problems and others are solved by the present invention which in one aspect is a flexible tube for use in medical procedures having a distal end and a body, as well as a visible marking of a predetermined color located on and substantially surrounding the tube along the length of the tube. The visible marking can be used to quickly identify the tube. In one embodiment, the visible marking is made up of a layer of suitable material. In another embodiment, visible marking can correspond to a predetermined fluid such as blood, a saline solution or the like.

In another aspect, the present invention is a medical apparatus for use in medical procedures having flexible tube with a distal end and a body, a visible marking of a predetermined color located on and substantially surrounding the tube (along the length of the tube) where the visible marking can be used to quickly and immediately identify the tube, and an intravenous bag fluidly coupled to the tube, where the intravenous bag is colored the predetermined color of the tube.

In another aspect, the present invention is a visible marking for identifying a medical tube, the visible marking being a colored collar. The colored collar has a distal end and body with an inner diameter capable of fitting around the exterior surface of the medical tube, where the colored collar helps a person quickly identify a color coded tube, intravenous bag or both. In one embodiment, the colored collar can be a semi-flexible material which extends around a portion of the exterior surface of the medical tube.

In yet another aspect, the present invention is system for administration of medical fluids to a patient, the system having (i) a first bag having a first color and containing a first fluid, and a first tube having the first color, the first tube fluidly coupled to the first bag, and (ii) a second bag having a second color and containing a second fluid, and a second tube having the second color, the second tube fluidly coupled to the second bag. Through the system of the present invention, the second color enables correlation between the second tube and the second bag. In other words, medical personnel and the like will be able to quickly assess the status of the patient and medication being administered quickly through use of the system. In one embodiment, the first bag is a first intravenous bag, the second bag is a second intravenous bag, the first fluid is a first medical fluid, and the second fluid is a second medical fluid.

In another aspect of the present invention, the system further has a third bag having a third color and containing a third fluid, and a third tube having the third color, the third tube fluidly coupled to the third bag, where the third color enables correlation between the third tube and the third bag. In one embodiment, the third bag is a third intravenous bag and third fluid is a third medical fluid.

In a final aspect, the present invention is a method of delivering fluids to a patient including the steps of: a) administering a first fluid to the patient, wherein the first fluid is contained within a first bag fluidly coupled to a first tube, the first bag and first tube having a first color; b) administering a second fluid to the patient, wherein the second fluid is contained within a second bag fluidly coupled to a second tube, the second bag and the second tube having a second color; and c) differentiating between the first fluid and the second fluid by correlating the first color with the second color.

In one embodiment, the method of the present invention further includes the step of administering a third fluid to the patient, where the third fluid is contained within a third bag fluidly coupled to a third tube, the third bag and third tube having a third color. In another embodiment, the first bag is a first intravenous bag, the second bag is a second intravenous bag, the first fluid is a first medical fluid, and the second fluid is a second medical fluid. In yet another embodiment, the third bag is a third intravenous bag and third fluid is a third medical fluid. In a final embodiment, the method of the present invention includes the step of identifying the type of fluid on the basis of its color.

### BRIEF DESCRIPTION OF DRAWINGS

FIG. 1 is a perspective cross-sectional view of tubing according to one aspect of the present invention.

FIG. 2 is a cross-sectional view of tubing according to one aspect of the present invention.

FIG. 3 is a perspective view of the collar engaging tubing according to one aspect of the present invention.

FIG. 4 is an isolated perspective view of the collar of one aspect of the present invention.

FIG. 5 is a front view of one aspect of the present invention.

FIG. 6 is a front view illustrating functional relationships between related elements according to one aspect of the present invention.

### DETAILED DESCRIPTION OF THE DRAWINGS

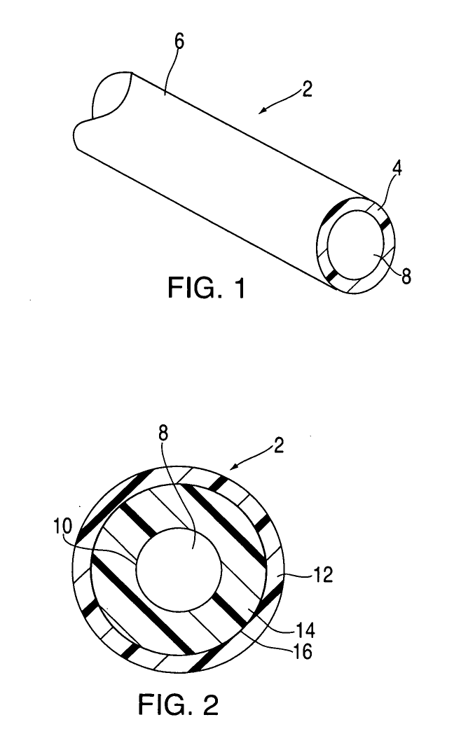

Referring to the drawings, FIG. 1 illustrates one embodiment of the present invention. A tubing 2 is shown having a distal end 4 and a body 6. The body 6 of the tubing 2 runs along a longitudinal axis and defines a passage 8 to allow for the movement of a predetermined fluid, such as a medical fluid, through such passage 8. The tubing 2 is preferably made of flexible or semi-flexible material including but not limited to a polymer, plastic, nylon, rubber or the like. In a preferred embodiment, the tubing 2 is based on conventional medical tubing used for the delivery of fluids to a patient. In addition, the tubing 2 may be comprised of a color or a pattern of multiple colors to achieve the objects of the present invention. One way to achieve such coloring is to add and mix, during the manufacturing stage, a safe dye into the raw material used to make the tubing 2.

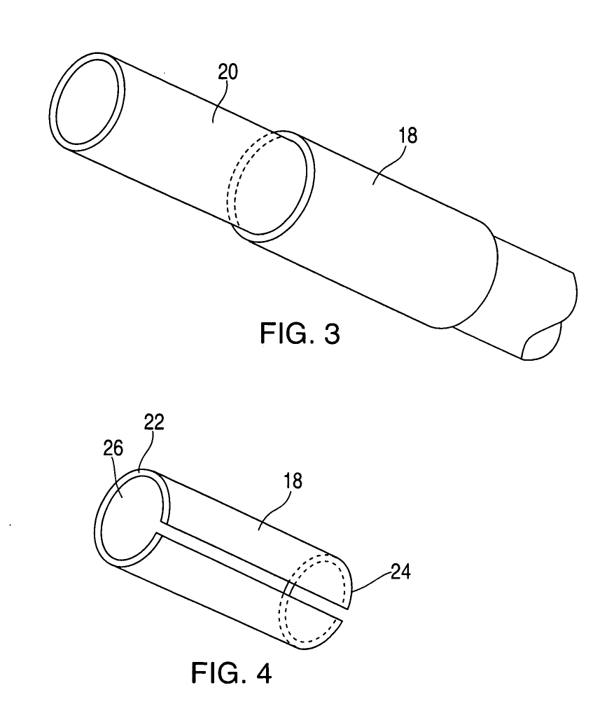

FIG. 2 illustrates a cross-sectional view of another embodiment of the tubing 2. The tubing 2 is generally circular in cross-section. The tubing 2 includes an inner tube 14 having an interior surface 10 and an exterior surface 16, and a visible marking 12 connected to the exterior surface 16 of the inner tube 14. The interior surface 10 defines a passage 8 that likewise has a generally circular cross-section, through which allows for the flow of fluid. For example, medical fluids capable of being administered through the tubing 2 include but are not limited to crystalloid solutions, dextrose solutions, Hartmann's solution (sodium lactate), colloids, dextrans, gelatins, hydoxyethyl starches (hetastarch), and blood.

The visible marking 12 is connected to the exterior surface 16 of the inner tube 14 and, in one embodiment, can extend along the length of the inner tube 14. In another embodiment, the visible marking 12 is connected to the exterior surface 16, but is only connected at certain portions along the length of the inner tube 14. For example, the visible marking 12 can extend around the entire circumference of exterior surface 16 of the inner tube 14 as a band of between 0.1 and 2 inches in length. The visible marking 12, can also be comprised of multiple bands separated by a distance ranging from a fraction of an inch to multiple inches along the length of inner tube 14. In another example, the visible marking 12 can extend only almost completely around the circumference of the inner tube 14, with the exception of a small gap (not shown). These examples, however, are neither meant to limit nor are inclusive of the embodiments of the configurations of the visible marking 12 with respect to the inner tube 14. Other configurations of visible marking 12 with respect to the inner tube 14 not mentioned are also included within the scope of the present invention. The visible marking 12 may be integrally manufactured with the inner tube 14, or the visible marking 12 can be attached to the inner tube 14 after the inner tubing 14 is manufactured.

The inner tube 14 can be made of flexible or semi-flexible material that is capable of withstanding the continuous flow of fluid through the passage 8. The flexible or semi-flexible material composing the inner tube 14 can include but is not limited to a polyurethane, polyamide, polyethylene, polypropylene, soft PVC, plastic, nylon, rubber or the like. The visible marking 12 can also be made of flexible or semi-flexible material including but not limited to polyurethane, polyamide, polyethylene, polypropylene, soft PVC, plastic, nylon, paper, rubber or the like. It is not necessary does not have the inner tube 14 and visible marking 12 to be made of the same flexible or semi-flexible material. Finally, the visible marking 12 can be comprised of a color of a combination of colors that allow users such as medical personnel to quickly and immediately verify the specific or type of fluid flowing through the tubing 2.

The inner tube 14 has a diameter, which in one embodiment ranges between about 5 and 30 millimeters. In a preferred embodiment, the diameter of the inner tube 14 is between about 6 and 20 millimeters. The wall thickness of the inner tube 14 varies in relation to the inner tube 14 diameter, and can range from about 1.5 to 5 millimeters. The visible marking 12 can have a corresponding wall thickness of between about 0.5 to 5 millimeters, depending on the wall thickness of the inner tube 14. Generally, the wall thickness of the visible marking 12 is less than that of the corresponding inner tube 14 wall thickness.

FIG. 3 illustrates another embodiment of the present invention, which comprises a colored collar 18 capable of connecting to a pre-existing conventional medical tubing 20 used for the delivery of fluids to a patient. As illustrated in FIG. 3, the colored collar 18 is affixed around the outer circumference of the conventional tubing 20. The colored collar 18 can be made of flexible or semi-flexible material including but not limited to polyurethane, polyamide, polyethylene, polypropylene, soft PVC, plastic, nylon, paper, rubber or the like. In one embodiment, the colored collar 18 is removably connected to a conventional medical tubing 20. In such a preferred embodiment, the color collar 18 is made from semi-flexible material which can be slidably removed from the conventional tubing 20, yet is capable of being secured to conventional tubing 20 during the administering of fluids to a patient.

In another embodiment, as illustrated in FIG. 4, the colored collar 18 is comprised of a semi flexible material having a gap 26 in the circumference of the collar 18, which gap 26 runs the entire length of the collar 18. The color collar 18 in this embodiment defines an opening which is slightly smaller than the diameter of the conventional tubing. As a result, the frictional forces between the outer surface of the conventional tubing and the inner surface of the color collar 18 should suitable secure the conventional tubing to the collar 18. The collar 18 can be removed from any conventional tubing in a number of ways, preferably, by sliding the collar 18 apart from the corresponding tubing along the length of the tubing. Another way includes flexing the edges along the length of the collar 18 such that the gap 26 of the collar 18 exceeds the diameter of the conventional tubing, thereby allowing the conventional tubing to be separated from the collar 18 via the gap 26.

The colored collar 18 can be of any desired length and can run along substantially the entire length of the corresponding conventional tubing 20. In another embodiment, the colored collar 18 can be comprised of multiple shorter portions having a first end 22 and second end 24, which individual portions are substantially shorter than the length of corresponding conventional tubing.

The colored collar 18 allows users such as medical personnel to quickly ascertain a particular or type of fluid flowing through the tubing. For example, a color collar 18 (having a particular color, e.g., blue) can be used in connection with a tube carrying D5W as opposed to normal saline (having another particular color, e.g., red). The color collar 18 serves not only to immediately identify the particular fluid being delivered to the patient but also the current condition of the patient. For example, using the color yellow to identify a dextrose solution that is used only on patients with diabetes would enable medical personnel to identify not only type of solution but the condition of the patient, i.e., diabetic. In addition, the colored collar 18 can be used in connection with pre-existing tubing already in use, since typically pre-existing tubing is clear or transparent. There is no need to dispose of current medical equipment, including tubing and the like when implementing the colored collar 18.

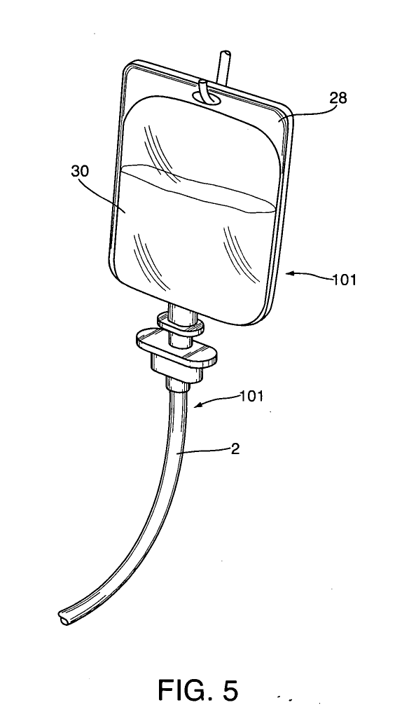

FIG. 5 illustrates the another aspect of the present invention, which includes a container and tubing 2. Preferably, the container is an intravenous bag 28. However, the container is not so limited and can also include (but is not limited to) a plastic bottle, a glass bottle, a plastic syringe, a glass syringe or the like, which may depend on the particular fluid being administered to the patient. In the embodiment illustrated in FIG. 5, the intravenous bag 28 and corresponding tubing 2 is of the same color 101. The color 101 can identify the particular fluid being delivered to the patient including but not limited to crystalloid solutions, dextrose solutions and the like. In addition the color 101 can identify the type of fluid to a patient including but not limited to chemotherapy-related medication, diabetes-related medication, and cardiac-related medication. For example, chemotherapy drug solutions administered intravenously may be linked or associated with a particular color or color pattern, which color or color pattern is distinct from that linked with diabetes-related solutions. Identification and verification of the fluids through color or color pattern may be of a primary and secondary resource in accurately and immediately assessing the condition of a patient in an emergency situation.

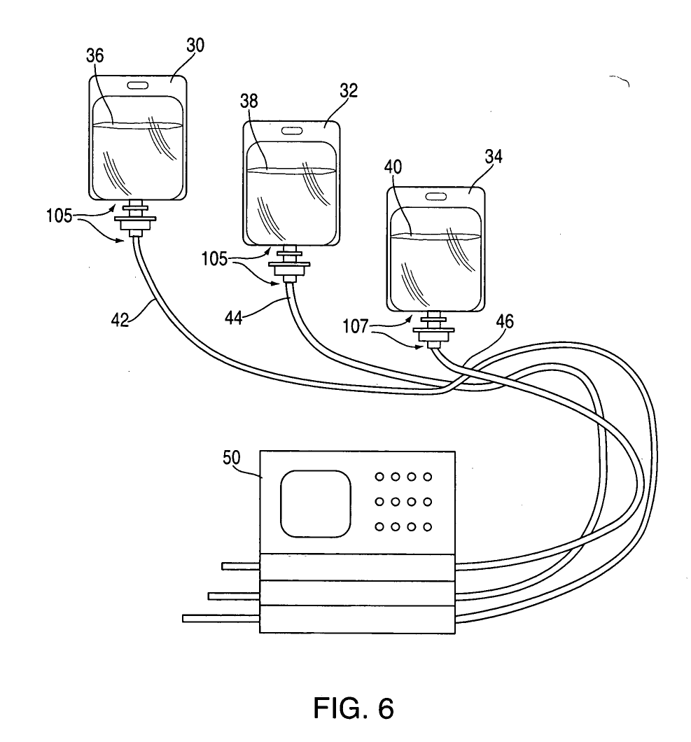

FIG. 6 illustrates functional relationships of elements according to one embodiment of the present invention. The system of the present invention generally comprises a first container 30 with a first tubing 42 (both of the first color 103), a second container 32 with a second tubing 44 (both of the second color 105) and a third container 34 with a third tubing 46 (both of the third color 107). The containers contain a first fluid 36, a second fluid 38 and a third fluid 40, respectively. In the preferred embodiment, the fluids 36, 38, 40 can be a medical fluid including but not limited to crystalloid solutions, dextrose solutions, Hartmann's solution (sodium lactate), colloids, dextrans, gelatins, hydoxyethyl starches (hetastarch), blood, plasma protein fraction (human albumin solution), and fresh frozen plasma. In another embodiment, the fluids 36, 38, 40 comprise gases including inhaled anesthetic gases such as halothane, sevoflurane and enflurane.

The system of the present invention provides for immediate differentiation among the tubing 42, 44, 46 and containers 30, 32, 34, and specifically among the particular medical fluids contained in the tubes and intravenous bags. The system of the present invention can also provide for accurate verification of the types of fluids 36, 38, 40 contain in the containers 30, 32, 34. In the case of high risk solutions such as chemotherapy drugs, such differentiation allows users such as medical personnel to readily verify the high risk solutions through the immediately recognizable colors and color patterns 103, 105, 107. The tubing and container colors or color patterns 103, 105, 107 also provide a secondary reference as, often times, medical personnel have to shift their focus from the fluids 36, 38, 40 being administered to the patient to the patient himself/herself to medical diagnostic equipment and then back to the fluids 36, 38, 40. In addition, the readily identifiable colors can aid in preventing the mixing of incompatible solutions within the tubing 42, 44, 46 and containers 30, 32, 34. Such a system assists in the prevention of mistakes by health-care personnel, which may in turn lead to better patient health care, lower insurance rates and lower risks of malpractice lawsuits.

It should be noted that certain embodiments do not include elements which can be operably used in connection with the elements disclosed in these embodiments. However, it is understood to one skilled in the art that such non-disclosed elements are generally well known and can be incorporated into these embodiment to allow for continued operation of the present invention.

Whereas the present invention has been described in relation to the accompanying drawings, it should be understood that other and further modifications, apart from those shown or suggested herein, may be made within the spirit and scope of the present invention. It is also intended that all matter contained in the foregoing description or shown in the accompanying drawings shall be interpreted as illustrative rather than limiting.

What is claimed is:

1. A flexible tube for use in medical procedures comprising:
a distal end and a body; and
a visible marking of a predetermined color located on and substantially surrounding the tube along the length of the tube, whereby the visible marking is adapted for identification of the tube.

2. The device of claim 1, wherein the visible marking comprises a layer of material.

3. The device of claim 2, wherein the visible marking corresponds to a predetermined fluid.

4. A medical apparatus for use in medical procedures comprising:
a flexible tube comprising a distal end and a body;
a visible marking of a predetermined color located on and substantially surrounding the tube along the length of the tube, whereby the visible marking is adapted for identification of the tube; and
an intravenous bag fluidly coupled to the tube, wherein the intravenous bag is colored the predetermined color of the tube.

5. A visible marking for identifying a medical tube comprising:
a colored collar having a distal end and body with an inner diameter capable of fitting around the exterior surface of the medical tube, wherein the colored collar enables identification of the tube.

6. The visible marking of claim 5, wherein the colored collar comprises a semi-flexible material which extends around a portion of the exterior surface of the medical tube.

7. A system for the administration of at least two medical fluids to a patient comprising:
a first container having a first color and containing a first fluid;
a first tube having the first color, the first tube fluidly coupled to the first container;
a second container having a second color and containing a second fluid; and
a second tube having the second color, the second tube fluidly coupled to the second container,
wherein the second color enables correlation between the second tube and the second container.

8. The system of claim 7, further comprising:
a third container having a third color and containing a third fluid; and
a third tube having the third color, the third tube fluidly coupled to the third container,
wherein the third color enables correlation between the third tube and the third container.

9. The system of claim 7, wherein the first container is a first intravenous bag, the second container is a second intravenous bag, the first fluid is a first medical fluid, and wherein the second fluid is a second medical fluid.

10. The system of claim 8, wherein the third container is a third intravenous bag and third fluid is a third medical fluid.

11. A method of delivering fluids to a patient comprising the steps of:
a) administering a first fluid to the patient, wherein the first fluid is contained within a first bag fluidly coupled to a first tube, the first bag and first tube having a first color;
b) administering a second fluid to the patient, wherein the second fluid is contained within a second bag fluidly coupled to a second tube, the second bag and the second tube having a second color; and
c) differentiating between the first fluid and the second fluid by correlating the first color with the second color.

12. The method of claim 11, further comprising the step of administering a third fluid to the patient, wherein the third fluid is contained within a third bag fluidly coupled to a third tube, the third bag and third tube having a third color.

13. The method of claim 11, further comprising the step of identifying a type of fluid on the basis of its color.

14. The method of claim 11, wherein the first bag is a first intravenous bag, the second bag is a second intravenous bag, the first fluid is a first medical fluid, and wherein the second fluid is a second medical fluid.

15. The method of claim 12, wherein the third bag is a third intravenous bag and third fluid is a third medical fluid.
